(19) United States
(12) Patent Application Publication
(10) Pub. No.: US 2008/0051376 A1
(43) Pub. Date: Feb. 28, 2008

(54) Method for Producing Albumin-Corticoid Conjugates

(75) Inventor: Hannsjorg Sinn, Wiesloch (DE)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Albupharm Heidelberg GMBH & Co. KG, Heidelberg (DE)

(21) Appl. No.: 11/632,443

(22) PCT Filed: Jul. 14, 2005
(86) PCT No.: PCT/EP05/07679

§ 371 Date: Jan. 12, 2007

(30) Foreign Application Priority Data
Jul. 14, 2004 (DE) 10 2004 034 008.0

(51) Int. Cl. A61K 31/56; A61P 35/00; A61P 37/06; C07K 14/00; C07K 14/76

(52) U.S. Cl. 514/169; 530/350; 530/362

(57) ABSTRACT

The invention relates to corticoid/transport protein conjugates, methods for the production thereof, and the use of the same in medicine, especially for the treatment of tumours and inflammatory processes, and for immunosuppression.

The present invention relates to corticoid-transport protein conjugates, methods for their production and their use in medicine, in particular for the therapy of tumors, of inflammatory processes and for immunosuppression.

Corticoids, also called corticosteroids, are steroid hormones of the adrenal cortex, which are formed under the influence of the hormone ACTH (corticotropin). Glucocorticosteroids such as, for example, cortisone, corticosterone and dexamethasone control the protein and sugar metabolism. Mineral corticoids, such as, for example, cortexolone, cortexone and aldosterone control the mineral metabolism.

It has been known for a long time that corticoids can influence numerous pathological processes in the body. Corticoids are therefore employed as a medicament in very different diseases, for example in order to suppress inflammation or to reduce immunological reactions. Corticoids can be employed therapeutically, for example, as antirheumatics, in arthritis, in allergies and in states of stress or shock.

Hitherto, various corticoids or their more water-soluble derivatives such as, for example, acetates, hemisuccinates, etc., were used for the treatment of inflammatory processes (Belgi. G. and Friedmann P. S. (2002): Traditionelle Therapien: Glukocorticoide, Azathioprin, Methotrexat, Hydroxy-Harnstoff [Traditional therapies: glucocorticoids, azathioprine, methotrexate, hydroxyurea]; H+G. Hautkrankheiten [skin diseases], Vol. 77, Issue 12, 624) and for immunosuppression in transplant rejections.

A disadvantage of the direct administration of corticoids is that only a very small amount of the active compound administered, in particular on systemic administration, reaches the target site on account of the ubiquitous whole body distribution. Thus, a higher dose is necessary, which especially in the case of relatively long administration leads to numerous undesired side effects such as, for example, hypertension, osteoporosis, general immunosuppression, induced diabetes mellitus, weight gain, muscular atrophy, skin changes such as acne and reduction of the faculty of the vision.

By coupling active compounds to endogenous proteins, it is possible to transport active compounds to certain sites in the body in order that these can display their action there. The covalent bonding of low molecular weight active compounds such as, for example, methotrexate to the macromolecule is described, for example, in DE 4122210 A1 or in WO 96/32133, where the conjugates formed there are employed for the treatment of oncoses or for the treatment of inflammation, infections and/or skin diseases. When using such conjugates for the treatment of oncoses, a tumor-active compound can thus be concentrated in tumor cells, whereas no increased absorption of the active compound bound to proteins takes place in healthy tissue.

An object of the present invention was to make available corticoid compositions in which the disadvantages observed in the prior art are at least partially overcome. In particular, corticoid compositions should be made available which have a long half-life in the body after administration and which also lead to no or only slight side effects on systemic administration.

This object is achieved according to the invention by corticoid-transport protein conjugates in which a corticoid is bonded covalently to a carrier protein.

By coupling to carrier proteins, the corticoids, which per se are removed rapidly from the body, are concealed from the excretion and/or capture mechanisms of the body and a long residence time in the organism is achieved. Owing to this prolonged half-life in the body, it is possible to reduce the amount of corticoid necessary and thus to suppress side effects which may occur. Moreover, the toxic action on healthy tissue or on organs can be virtually avoided, since normal cells have no reason for protein uptake. Advantageously, the corticoid is released from the conjugate according to the invention only at the target site, such that lower doses per kg of body weight are often adequate. Thus the burden on the liver and the other healthy organs is further reduced.

According to the invention, a carrier protein is a protein which functions as a carrier molecule for the corticoid active compound. Examples of such proteins are those proteins having a molecular weight of ≧18 000 Da, more preferably ≧50 000 Da, in particular 100 000 Da. Advantageously, the corticoid active compound can be brought selectively to certain sites in the body by the carrier protein. In this way, it is achieved that smaller corticoid doses are sufficient in order to obtain a desired action and the customary side effects which can occur on systemic administration of corticoids in a high dose are thus suppressed.

According to the invention, proteins in native form which are regarded as not exogenous are preferably used as carriers. More favorably, depending on the patient to whom the conjugate is to be administered, an appropriate native protein is selected, for instance a human protein for administration to humans.

Suitable proteins are, for example, transferrin and preferably albumin, more preferably serum albumin and most preferably human albumin (HSA; human serum albumin). With a molecular weight of approximately 68 kDa, albumin is the smallest of the proteins occurring in the plasma. However, it makes up approximately 60% of the total amount of plasma protein. As an endogenous, ubiquitously distributed nonimmunogenic protein, albumin fulfills, inter alia, transport functions for many substances in the healthy organism and serves in an acute emergency as a reserve energy carrier, which is available anywhere and at any time. Since albumin is not absorbed by healthy cells, its use is advantageous in the corticoid-transport protein conjugates according to the invention.

Owing to the covalent coupling according to the invention of the corticoid to the carrier molecule, it is achieved that no restriction of the native character of the protein takes place. Thus the conjugate according to the invention is not regarded as exogenous, and can remain in the organism for a longer time.

The conjugates of the invention preferably contain a corticoid and a transport protein in the molar ratio of 2:1 to 0.5:1, more preferably the molar ratio of corticoid to transport protein is 1.1:1 to 0.9:1. In particular, a molar ratio of approximately 1:1 is advantageous. Here, bonding can take place either directly, in linker-free form or by means of a linker to the transport protein.

A linker-free bonding of the corticoid to the carrier means that the corticoid is bonded to the transport protein by a direct chemical bond. For example, the corticoid can be bonded covalently to the protein by means of an ester group which is formed from an OH group of the corticoid and an acid group of the protein.

According to one aspect of the present invention, a bond via a linker is preferred. Linker in the sense of the invention means a structural unit by means of which a corticoid is bonded to a transport protein. Particularly suitable linker molecules contain, for example, 2 acid groups or 2 activated acid groups by means of which coupling can take place, on the one hand to the corticoid and on the other hand to the protein. An example of a particularly suitable linker is ethylenediaminetetraacetic acid (EDTA).

It is important with the type of coupling that the covalent bond can be cleaved again in the target cell in order that the corticoid can be released again there and can display its biological activity. Cleavage is carried out by means of a chemical change in the bonding site to the linker. In the case of bonding of the corticoid and of the carrier protein to the linker by means of ester groups, the ester bond can be cleaved again in the target cell, for example by enzymatic ester cleavage.

According to the invention, any desired corticoids can be bonded to a carrier protein. In addition to naturally occurring and synthetically prepared steroid hormones of the adrenal cortex, the designation “corticoid” in the sense of the invention also comprises compounds having a corticoid structure, in particular steroid antibiotics. According to the invention, compounds which are derived from the tetracyclic hydrocarbon perhydro-1H-cyclopenta[A]-phenanthrene(sterane) are preferred. Compounds preferably employed have the formula I 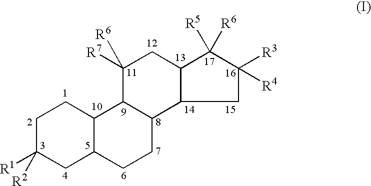
where these are optionally unsaturated in the 1, 2 and/or 4, 5 position where one of more of the positions 1, 2, 4-10 and 12-15 can in each case independently be substituted by one or two radicals R⁹, and in which

R¹ and R² can together be O or R¹ is OH and R² is H;

R⁷ and R⁸ can together be O or R⁷ is OH and R⁸ is H;

R³-R⁶ and R⁹ are in each case independently selected from H, OH, halogen, C₁₋₄-alkyl, and can contain a monovalent radical of the heteroatoms, in particular N, O, P and S;

R⁵ and R⁶ can together form a divalent radical which can be mono- or polyunsaturated and/or can contain hetero-atoms, in particular N, O, P and S;

and where at least one of the radicals R¹-R⁹ is and/or contains a functional group such as, for example, —OH or amine.

Preferred radicals R³ are H, OH, Cl, F and/or CH₃ and/or they can preferably have the formula (II) 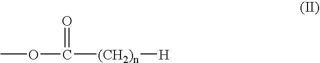
in which n is an integer from 0-4. Most preferably, n=1.

Preferred radicals R⁵ are H, OH, Cl, F and/or CH₃ and/or they can preferably have the formula (III) 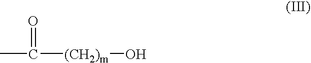
in which m is an integer from 0-4.

Most preferably, m=1.

Preferred radicals R⁴, R⁶ and R⁹ are H, OH, CH₃, Cl, and/or F and/or they can preferably have the formula (IV) 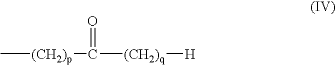
in which p and q independently of one another can be an integer from 0-4.

Preferably, p and q are in each case 0.

If R⁵ and R⁶ together form a divalent radical, this preferably has the formula (V), 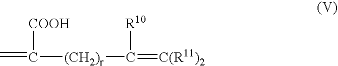
in which r can be an integer from 0-4 and R¹⁰ and R¹¹ are in each case independently selected from H and C₁-C₄-alkyl.

Most preferably, r=2.

R¹⁰ is preferably H and

R¹¹ is preferably CH₃.

Preferably, a human corticoid is used. Examples of corticoids advantageously employed are hydrocortisone, cortisone, triamcinolone, cortisone acetate, cloprednol, aldosterone, prednisole, prednisolone, fluocortolone, triamcinolone, methylprednisolone, betamethasone, desoxymethasone, clobetasone butyrate, hydrocortisone butyrate, fluocinolone acetonide, prednicarbate, triamcinolone acetonide, halcinoid, betamethasone dipropionate, betamethasone valerate, diflorasone diacetate, difluocortolone valerate, clobetasol propionate, corticosterone and dexamethasone is very particularly preferably employed. Particularly more recently, synthetically prepared dexamethasone is preferably used. A further preferred compound of the formula I is the corticoidal antiinflammatory fusidic acid.

According to a further aspect, the present invention makes available a method for the production of corticoid-transport protein conjugates. In the method according to the invention, a corticoid and a transport protein are reacted with one another, and linkage by means of covalent bonds takes place in the reaction.

According to the invention, one possibility for production is the direct coupling of corticoid and transport protein. For example, by reaction of a carboxyl group of the corticoid (e.g. fusidic acid) with an amino group of a protein side chain a linkage can take place with formation of an amide group. In a direct coupling, the time-consuming production and workup of intermediate products is superfluous.

In a further preferred embodiment of the method of the present invention, a corticoid and a transport protein are reacted with a linker, and linkage by means of covalent bonds takes place in the reaction. In this method, the linker favorably contains two functional groups by means of which bonding can take place on the one hand to the corticoid and on the other hand to the transport protein. Such functional groups on the linker molecule can be, for example, activated carboxylic acid groups such as anhydride groups, carboxylic acid chlorides and the like. A particularly suitable linker in the method according to the invention is EDTA dianhydride.

In the linkage of the corticoid to a carrier protein by means of a linker, a crosslink reaction can take place as a side reaction. “Crosslink reaction” is understood in the sense of the invention as meaning the linkage of a number of corticoids/proteins by means of linkers. Such relatively large conjugates are less suitable for medicinal use, since they are eliminated more rapidly from the circulation and lead to antibody formation. According to the present invention, crosslinking can be avoided by adding an ammonia solution in a further step.

In order that the corticoid contained in the conjugate according to the invention shows its full activity, cleavage of the linker and if appropriate breakdown of the protein still bonded to the linker must take place in the target cell. The linkers selected are therefore preferably those compounds which can be removed again in a respective target cell. It is known to the person skilled in the art by means of which factors, e.g. enzymes, the cleavage of certain chemical bonds can take place in cells. For example, ester groups can be cleaved by means of enzymatic ester cleavage by esterases. Acid amide bonds can be cleaved by enzymatic peptide cleavage.

The conjugates according to the invention are distinguished in that they can be transported selectively to certain sites in the body and thus the corticoid can be enriched there. The corticoid can be released and display its activity only at the target site. It has been found that the conjugates according to the invention are absorbed to an increased extent by tumor cells. In contrast to this, healthy cells do not absorb the conjugates or only absorb them to a significantly smaller extent. The conjugates according to the invention are therefore outstandingly suitable for therapeutic purposes, in particular for the therapy of oncoses such as, for example, solid tumors.

Surprisingly, it has been found that the conjugates according to the invention are absorbed not only in tumor cells but also in cells relevant for immune reactions. Thus an enrichment of active compound also takes place in these cells. Corticoids have an inhibitory action on the expression of a very large spectrum of proinflammatory and immunoregulatory cytokines such as interleukin (IL), interferon, tumor necrosis factor TNF-α and of a number of costimulatory factors. The conjugates of the present invention are consequently also suitable for the suppression of immune reactions, for example in transplantation-associated immune reactions. This action opens up a wide spectrum of use for the conjugates according to the invention for the avoidance of immunological complications in transplantation, in particular in the case of allogenic or autologous bone marrow transplants but also for the avoidance of recipient-mediated rejection reactions in organ transplants, in particular in foreign donor organ transplants of, for example, kidney, heart or liver. The conjugates according to the invention can therefore be employed advantageously for the treatment and/or prophylaxis of GVHD and in particular of acute or chronic GVHD.

A particular advantage of the conjugates according to the invention for use in connection with undesired immune reactions is that locally restricted immuno-suppression is made possible, since essentially no absorption of the conjugates by healthy cells takes place.

An advantage of the use of the conjugates according to the invention is their long residence time in the organism. In general it is ≧15 days and preferably ≧19 days. Thus, in comparison to the hitherto customary direct administration of corticoids a reduction of the required dose which is necessary in order to achieve a desired action is made possible. According to the invention, the amount of corticoid administered, in particular dexamethasone, can be, for example, from 0.1 μg/kg of body weight to 0.1 g/kg of body weight, in particular from 10 μg/kg of body weight to 0.01 g/kg of body weight.

### FIGURES

FIG. 1 shows the chromatogram of the HPLC investigation of the reaction products of the inventive example. Free dexamethasone is detected after a retention time of 31.45 min.

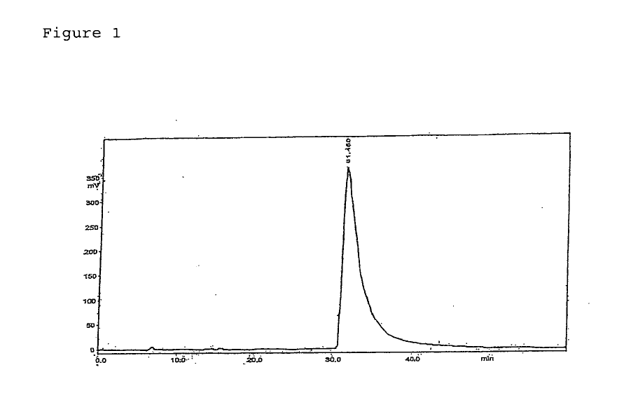

FIG. 2 shows the chromatogram of the HPLC investigation of the inventive example, a dimeric albumin fraction being detected after 7.07 min and a monomeric albumin fraction being detected after 8.27 min.

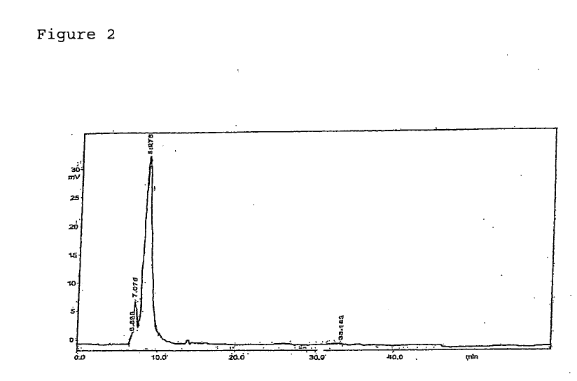

### INVENTIVE EXAMPLE

20 mg of dexamethasone (MW 392.5 g/mol) are initially introduced together with approximately 14 mg of EDTA dA (MW 256.22 g/mol) into a test-tube having an NS 14.5 ground glass joint and stopper. After the addition of 2 ml of pyridine, the reaction mixture is introduced into a water bath preheated to 650C. After a reaction time of 6 h, a colorless, clear solution is present which, after cooling to room temperature, is drawn into a glass syringe and very slowly introduced into a 5% strength albumin solution. Turbidity is briefly formed at the inflow site which, however, rapidly resolves again. Shortly after the end of the addition of active compound, 0.5 ml of a 56 strength ammonia solution is added for the avoidance of any possible cross-link reactions.

Reaction control of the reaction of dexamethasone with EDTA dianhydride by means of thin layer chromatography (TLC)

1 μl of the original solution is applied to a TLC aluminum foil 5×10 cm, silica gel 60, F₂₅₄ (E. Merck) and developed in a TLC chamber using 0.33% strength methanolic acetic acid.

| | | |
|---|---|---|
| Rf values: | dexamethasone | 0.88-0.9 |
| | dexamethasone EDTA | 0.48-0.5 |

Quality Control (HPLC):

| | |
|---|---|
| precolumn: | LiChrospher 100 DIOL 5μ (25 × 10 mm) |
| | (Besta-Technik) |
| column: | LiChrospher 100 DIOL 5μ (25 × 10 mm) |
| | (Besta-Technik) |
| eluent: | 0.2 M Na citrate, pH 7.4 |
| flow: | 1.0 ml/min |
| pressure: | approximately 51 bar |
| UV-vis: | 280 nm |

Retention Times:

| | |
|---|---|
| dimeric albumin fraction: | 7.07 min |
| monomeric albumin fraction: | 8.27 min |
| free dexamethasone: | 31.45 min |

The fraction of dimeric albumin is <5%, which means that negligible crosslinking has taken place during the loading. This is of essential importance for the avoidance of a rapid elimination from the circulation.

1. A corticoid-transport protein conjugate, comprising a corticoid covalently bonded to a carrier protein.

2. The corticoid-transport protein conjugate as claimed in claim 1, characterized in that
the protein is present in native form.

3. The corticoid-transport protein conjugate as claimed in claim 1, characterized in that
the protein is albumin.

4. The corticoid-transport protein conjugate as claimed in claim 1, characterized in that
the molar ratio corticoid:transport protein is 2:1 to 0.5:1.

5. The corticoid-transport protein conjugate as claimed in claim 4, characterized in that
the molar ratio corticoid:transport protein is 1.1:1 to 0.9:1.

6. The corticoid-transport protein conjugate as claimed in claim 1, characterized in that
the corticoid is bonded to the carrier protein in linker-free form.

7. The corticoid-transport protein conjugate as claimed in claim 1, characterized in that
the corticoid is bonded to the carrier protein by means of a linker.

8. The corticoid-transport protein conjugate as claimed in claim 7, characterized in that
the corticoid is bonded to the linker by means of an ester group and the linker is coupled to the carrier protein by means of an amide bond.

9. The corticoid-transport protein conjugate as claimed in claim 8, characterized in that
the ester bond is cleavable by enzymatic ester cleavage and/or the amide bond is cleavable by means of enzymatic peptide cleavage.

10. The corticoid-transport protein conjugate as claimed in claim 7, characterized in that
the linker is EDTA.

11. The corticoid-transport protein conjugate as claimed in claim 1, characterized in that
the corticoid is human corticoid.

12. The corticoid-transport protein conjugate as claimed in claim 1, characterized in that
the corticoid is dexamethasone.

13. The corticoid-transport protein conjugate as claimed in claim 1, characterized in that
the corticoid is fusidic acid.

14. A method for producing a corticoid-transport protein conjugate as claimed in claim 1, characterized in that
a corticoid and a transport protein are reacted with one another, and in the reaction a linkage by means of covalent bonds takes place.

15. A method for producing a corticoid-transport protein conjugate as claimed in claim 7, characterized in that
a corticoid and a transport protein are reacted with a linker, and in the reaction a linkage by means of covalent bonds takes place.

16. The method as claimed in claim 15, characterized in that
the linker has two activated carboxylic acid groups.

17. The method as claimed in claim 16, characterized in that
in a first step the corticoid is reacted with an activated carboxylic acid group of the linker and in a second step coupling of the protein to a further activated carboxylic acid group of the linker takes place.

18. The method as claimed in claim 16, characterized in that
the activated carboxylic acid groups are anhydride groups.

19. The method as claimed in claim 15, characterized in that
EDTA dianhydride is used as a linker.

20. The method as claimed in claim 15, characterized in that
after the coupling an ammonia solution is added in a further step.

21. A method for treating a tumor in a patient comprising the step of administering a therapeutically effective amount of the corticoid-transport protein conjugate of claim 1, wherein the corticoid is a compound active in treating the tumor in the patient.

22. The method as claimed in claim 21, wherein the tumor is a solid tumor.

23. A method for treating an inflammation process in a patient, comprising the step of administering a therapeutically effective amount of the corticoid-transport protein conjugate of claim 1, wherein the corticoid is a compound active in treating the inflammatory process in the patient.

24. A method for immunosuppression in a patient, comprising the step of administering a therapeutically effective amount of the corticoid-transport protein conjugate of claim 1, wherein the corticoid is a compound active in suppressing an immune response in the patient.

25. A method for suppression of an inflammatory process, immune response or tumor growth in a patient in need thereof, comprising the step of administering a therapeutically effective amount of the corticoid-transport protein conjugate of claim 1, wherein the corticoid is a compound active in suppressing an inflammatory process immune response or tumor growth in the patient, characterized in that
the corticoid is released from the conjugate at a target site within the patient.

26. The method of claim 25, characterized in that
a biological half-life of the conjugate is more than 15 days.

27. The method of claim 25, characterized in that
the concentration of corticoid in a healthy tissue is not increased compared to that in an unhealthy tissue or diseased cell.

28. The method of claim 24, wherein the immunosuppression comprises suppression of transplant rejection.
